(19) United States
(12) Patent Application Publication
(10) Pub. No.: US 2022/0249548 A1
(43) Pub. Date: Aug. 11, 2022

(54) APPLICATION OF CARBON-BASED NANOMATERIAL IN PREPARATION OF DRUG FOR RELIEVING OR TREATING HD

(71) Applicant: SOOCHOW UNIVERSITY, Suzhou (CN)

(72) Inventors: Zaixing YANG, Suzhou (CN); Xiuhua YIN, Suzhou (CN); Zhenhui KANG, Suzhou (CN); Ruhong ZHOU, Suzhou (CN)

(21) Appl. No.: 17/627,810

(22) PCT Filed: Jul. 17, 2020
(86) PCT No.: PCT/CN2020/102875

§ 371 Date: Jan. 17, 2022

(30) Foreign Application Priority Data
Jul. 18, 2019 (CN) 201910652242.1

(51) Int. Cl. A61K 33/44

(52) U.S. Cl. CPC A61K 33/44; B82Y 5/00

(57) ABSTRACT

Application of carbon-based nanomaterial in the preparation of drug for alleviating or treating HD. The carbon-based nanomaterial was prepared from the vitamin or quasi-vitamins.

### TECHNICAL FIELD

The invention belongs to nano-medicine technology, in particular to the application of carbon-based nano material in the preparation of drug for alleviating or treating HD.

### BACKGROUND TECHNIQUE

Huntington's disease (HD) is a late-onset autosomal dominant neurodegenerative disease. The main pathological features are extensive neuronal dysfunction and selective striatal neuronal degeneration, which is characterized by severe destruction of small ganglion cells, accompanied with glial proliferation, prominent pathological manifestations and atrophy of cortex.

There is a CAG trinucleotide repeat sequence in the first exon of HD gene. The encoded product is a polyglutamine fragment (Poly-Q) at the N-terminus of Htt. In the normal population, the number of CT repetitions of the HD gene is less than 35, and normal Htt (WT) is diffusely distributed in the cells. The mutant HD gene encodes a mutant huntingtin (mutanthuntingtin, mHtt) with an ultra-long (Poly-Q) structure and misfolded. Studies have shown that the age of onset of HD and the severity of HD are related to the length of poly-Q. mHtt exists widely in the nucleus and cytoplasm in dissociated and aggregated forms, misfolding and causing cytotoxicity, impairing the normal physiological functions of neurons, and leading to HD neuropathological damage. The misfolding of mutant Htt is the material basis of HD neuropathological damage, so inhibiting its formation or promoting its clearance is of great significance to delaying the pathological process of HD.

The application of nanotechnology in the diagnosis, mitigation and treatment of diseases is the rapidly developing and very promising field, but it is still in its infancy. Nanomaterials play an extremely important role as a potential nanomedicine for the diagnosis, relief and treatment of HD. In the past few years, research on the use of passive and active transportation of nanoparticles to deliver drugs to the brain has made great progress. Although people have great hopes for nanomaterial drugs as “smart” drugs and used in HD treatment, the cause of HD has not been fully elucidated, and the difficulty of HD treatment drugs to penetrate the blood-brain barrier brings difficulties to HD treatment. Throughout various studies, finding HD diagnosis and effective intervention methods requires courage and innovative thinking, and thee tireless pursuit of researchers. In addition, unlike the neurodegenerative diseases (AD, PD) caused by the aggregation of the other two major types of proteins, the Htt protein that causes the onset of HD is accumulated in the cell or even in the nucleus. It increases the difficulty of treating HD with drugs that target protein aggregation. Such drugs not only need to have the function of penetrating the blood-brain barrier, but also need to have the function of penetrating cells and being able to enter the nucleus.

### Technical Problem

The invention discloses an application of carbon-based nanomaterial in the preparation of drug for relieving or treating HD. The carbon nanomaterial is a new carbon nanomaterial discovered after fullerene, carbon nanotube and graphene. It is a quasi-spherical nanoparticle with a size of less than 10 nm with good water solubility, biocompatibility, fluorescence stability, stable physical and chemical properties, easy to realize surface functionalization, and can inhibit the accumulation or elimination of mHtt (mutant huntingtin, also known as mutant Htt) to achieve HD prevention.

### Technical Solutions

The present invention adopts the following technical solutions:

The invention discloses the application of carbon-based nanomaterials in the preparation of mHtt aggregation inhibitors or scavengers, or the application of carbon-based nanomaterials in the preparation of drugs for treating or relieving HD.

The invention also discloses the application of the carbon-based nano material in inhibiting the accumulation of mHtt or removing mHtt.

The invention also discloses a method for inhibiting the aggregation of mHtt, which includes the following steps: incubating the aqueous solution of carbon-based nanomaterials and the mHtt monomer to achieve the inhibition of mHtt aggregation.

The preparation method of the carbon-based nano material of the present invention includes the following steps: vitamins or quasi-vitamins are used as raw materials, and the carbon-based nano material is prepared through a heating reaction. mHtt is the mutant huntingtin protein, which can also be called mutant Htt. HD is Huntington's disease.

In the above, the vitamin solution or quasi-vitamins solution is at from 170° C. to 190° C. for 1.5 h to 2.5 h; then it is naturally cooled to room temperature, and then filtered; then the filtrate is dialyzed and lyophilization to obtain carbon-based nanomaterials, called CDs.

In the above, the concentration of vitamin solution is 0.1 g/ml; The concentration of quasi-vitamins solution is 0.1 g/ml; vitamins include vitamin A, vitamin E, vitamin D3, vitamin B1, vitamin B2, vitamin B6, vitamin C, vitamin K3, vitamin B12, etc.; quasi-vitamins are retinoid, quasi-vitamins D3, quasi-vitamins E, etc.

In the above, the vitamin solution is reacted at 180° C. for 2 hours, and the vitamins are polymerized to produce water-soluble carbon nanomaterials.

In the above technical scheme, the 500 to 1000 Da dialysis bag is used for dialysis; the dialysis is performed in water. The filtrate is an aqueous solution of carbon-based nanomaterials, which can be used directly to inhibit the accumulation of mHtt or to remove mHtt; it can also be lyophilization to obtain carbon-based nanomaterials and then reconstituted for use.

In the above technical solution, lyophilization is carried out at −80° C. and vacuum degree of 10 Pa for 48 hours. Preferably, lyophilization is performed by freezing in a refrigerator at −80° C. for 2 hours, and then lyophilization in the freeze dryer at −80° C. with the vacuum of 10 Pa for 48 hours.

Carbon sources of carbon quantum dots include carbon-based materials such as graphite-structured carbon materials and multi-walled carbon nanotubes. However, its expensive raw materials and the required high-energy systems limit its production and application. Natural organisms, such as grapefruit peel, orange juice, etc. can also prepare carbon quantum dots, but these substances are complex in composition and contain many impurities, which are not conducive to analysis. And due to the large individual differences of natural organisms, it is difficult to repeat the technical effects.

The present invention also discloses drugs for inhibiting mHtt aggregation, drugs for removing mHtt or drugs for treating HD, including the above-mentioned carbon-based nanomaterials. Treatment includes its generally accepted meanings, such as preventing, relieving, inhibiting, ameliorating and slowing down or stopping reversing the development of symptoms or expected lesions. The invention encompasses therapeutic and alleviating properties.

The medicament of the present invention may also include at least one of a pharmaceutically acceptable carrier, a pharmaceutically acceptable diluent, and a pharmaceutically acceptable excipient. The drug form can be tablet, pill, powder, tablet, small capsule, flat capsule, elixir, suspension, emulsion, solution, syrup, aerosol, ointment, soft and hard gelatin capsule, suppository, sterile injection solution or sterile packaging powder injection. In the invention, the active ingredient carbon based nano material is prepared into a drug or pharmaceutical composition, which can be prepared by a method known to those skilled in the field, so that it can release the active ingredient quickly, slowly or delayed after being applied to the subject. For example, the active ingredient can be mixed with the carrier (normal saline, buffer, etc.) and diluted or encapsulated in the carrier; Some substances suitable as carriers, excipients and diluents can be exemplified as lactose, dextrose, sucrose, sorbitol, mannitol, starch, resin, Arabic gum, calcium phosphate, alginate, tragacanth gum, gelatin, calcium silicate, microcrystalline cellulose, polyvinylpyrrolidone, cellulose, water syrup, methylcellulose, Methyl Paraben and propyl ester, talc powder, magnesium stearate and liquid paraffin. The medicine of the invention can also include lubricants, wetting agents, emulsifying and suspending agents, preservatives, sweeteners or flavoring agents and other additives.

Preferably, the drug of the present invention is a liquid, such as an aqueous solution of carbon-based nanomaterials. More preferably, the concentration of the carbon-based nanomaterial in the liquid drug is form 0.01 to 1 mg/mL (the concentration of the CDs aqueous solution in FIG. 4 reaches 70 mg/mL), preferably, from 0.025 to 0.5 mg/mL. The water is water for injection.

Inhibiting the accumulation of mHtt or eliminating mHtt is the key to HD treatment. However, HD is a long neurodegenerative disease. Whether the currently reported nanomaterials/drugs can finally be used in the clinic is not only determined by their mitigation and treatment effects, but also on their biotoxic effects and in vivo safety. HD is a kind of central nervous system disease. Whether drug molecules can pass through the blood-brain barrier in a noninvasive way is the prerequisite. The carbon-based nano material disclosed in the present invention has the advantages of small particle size, large specific surface area, surface functional group modification, low toxicity and degradability, and can pass through the blood-brain barrier, especially, can penetrate cells and enter the nucleus to inhibit the accumulation of mHtt or (partially) eliminate mHtt. It is a carbon-based nanomaterial which is effective for alleviating and treating HD.

### DESCRIPTION OF THE DRAWINGS

FIG. 1 shows the structural characteristics of CDs, (a) is X-ray photoelectron spectroscopy, (b) is infrared spectrum of CDs.

FIG. 2 (a) is the ultraviolet-visible absorption spectrum of the CDs aqueous solution, (b) is the spectral properties of the CDs aqueous solution.

FIG. 3 shows the morphology of CDs, (a) is transmission electron microscope (TEM) morphology observation, (b) shows hydrated particle size distribution, (c) is the height measured by atomic force microscope.

FIG. 4 is photos of CDs aqueous solutions with different concentrations.

FIG. 5 shows the results of CDs entering the nucleus, all with a 20 μm scale; (a) the electron micrograph of the normally cultured cells, (b) the electron micrograph of the cells incubated with C2N, and (c) is the edge exosomes of (b) Enlarged image, (d) 405 nm confocal image after co-incubation with CDs, (e) Red dotl stained nucleus image, (f) bright field image of the cell, (g) combined image of the three channels.

FIG. 6 shows CDs inhibiting the aggregation of mHttQ120 (abbreviated as Q120) polypeptides, (a) Th T fluorescence test to detect the content of β sheets, (b) to detect fiber production by dot hybridization experiment, (c) to aggregates (Q120, Q120+CDs) for morphological observation.

FIG. 7 shows the secondary structure of the mHttQ120 polypeptide aggregation product detected by circular dichroism.

FIG. 8 shows the CDs improve the survival rate of N2a cells transfected with mHttQ120 (a) lactate dehydrogenase experiment, (b) trypan blue staining, (c) live/dead cell staining statistics, (d) live/dead cell staining experiment graph (*P<0.05, **P<0.01).

FIG. 9 shows the cytotoxicity of CDs on SH-SYSY, PC12 cell lines, primary neurons and primary astrocytes detected by CCK8.

FIG. 10 shows the CDs erythrocyte lysis experiment, (a) is the real shots of different concentrations of CDs and red blood cells are incubated, (b) shows the release rate of heme in the supernatant detected by the microplate reader after incubation at 540 nm.

FIG. 11 shows how CDs can improve the life expectancy, weight loss and exercise ability of HD transgenic mice. (a) Survival curve of mice in each group; (b) comparison of body weight of mice in each group at 14 weeks; (c)) is the rotation axis experiment of HD mice; (d) is the lanyard endurance test of HD mice (*P<0.05, **P<0.01).

FIG. 12 shows the immunofluorescence experiment to detect the aggregation of mHtt in the cortex and striatum of each group of HD mice. The red is the staining of mhtt protein antibody (MW8), and the blue is the nuclear DAPI staining.

FIG. 13 is a diagram showing the effect of CDs in inhibiting the aggregation of mHttQ120 polypeptide in Example 5.

FIG. 14 is a graph showing the inhibitory effect of comparative carbon material on the aggregation of mHttQ120 polypeptide.

### EMBODIMENTS OF THE INVENTION

In neuropathology, CGA trinucleotide repeat sequence was found in the first exon of the HD gene, and its encoded product is a polyglutamine fragment (Poly-Q) at the N-terminus of Htt. The mutant HD gene encodes a mutant huntingtin (mutanthuntingtin, mHtt) with an ultra-long (Poly-Q) structure. In the normal population, the number of CAG repeats in the HD gene is less than 35. Mutation Htt misfolds, and exists widely in the nucleus and cytoplasm in free and aggregate forms, causing cytotoxicity, impairing the normal physiological functions of neurons, and leading to HD neuropathological damage. The misfolding of mHtt is the material basis of HD neuropathological damage. Therefore, inhibition of mHtt accumulation or elimination of mHtt is an important strategy to alleviate and treat HD. The carbon-based nano material disclosed in the present invention has the advantages of small particle size, large specific surface area, surface functional group modification, low toxicity and degradability, etc., and can pass through the blood-brain barrier, especially can penetrate cells and enter the nucleus. mHtt accumulates or removes mHtt, and is a carbon-based nanomaterial that is effective for HD relief and treatment.

The preparation method of the carbon-based nanomaterial of the present invention is as follows: the vitamin solution or quasi-vitamins solution react at 170° C. to 190° C. for 1.5 h to 2.5 h; and then natural cooling to room temperature and filter; the filtrate is dialyzed and lyophilization to obtain Carbon-based nanomaterial, called CDs.

The description of specific exemplary embodiments of the present invention is for the purpose of illustration and illustration. These descriptions are not intended to limit the invention to the precise form disclosed, and it is obvious that many changes and varieties can be made in accordance with the teachings of the invention. The purpose of selecting and describing the exemplary embodiments is to explain the specific principles of the present invention and its practical application, so that those skilled in the art can realize and use various exemplary embodiments of the present invention and multiple options. The scope of the present invention is intended to be defined by the claims and their equivalents.

### EXAMPLE 1

Preparation of Carbon-Based Nanomaterials (CDs)

Weighed 1.00 g of left-handed vitamin C (L-Vc) and dissolved it into 10 mL of H2O. Ultrasound for 20 minutes to complete dissolution; transfer the dissolved Vc solution to a hydrothermal kettle and reacted at 180° C. for 2 h. After the end, natural cooling to room temperature, then filter the reaction solution with a funnel to remove insoluble particles, and then purified with the 500 to 1000 Da dialysis bag in water, finally obtained the CDs solution was in brown-red; frozen the brown-red CDs solution in the refrigerator at −80° C. for 2 h, and then lyophilization at −80° C. with vacuum of 10 Pa for 48 h with an Alpha1-4LSCplus RC6 freeze dryer to obtain carbon-based nanomaterial CDs. The obtained carbon-based nanomaterial CDs were dissolved in pure water to obtain the aqueous solution of carbon-based nanomaterial CDs, which was used in Examples 2 to 4.

Ultraviolet spectrum: Diluted the CDs aqueous solution to a certain concentration and shifted into the cuvette, and measured with the ultraviolet spectrometer. Preparation and photography of electron microscopy sample: picking up the copper mesh with a tweezers in advance and placed it on absorbent filter paper, dropped the 5 μLCDs aqueous solution on the copper mesh, and placed it in the cool place to air dry. After the sample was dried, taken the photos with the FEI Tecnai G20 electron microscope, and the high magnification pictures were taken with the JEM-2010F high transmission electron microscope.

X-ray photoelectron spectroscopy analysis of CDs nanomaterial: taken some powder samples of carbon-based nanomaterial CDs and tested on the X-ray photoelectron spectrometer;

Determination of infrared spectrum: taken some powder samples of CDs lyophilization and tested on the infrared spectrometer.

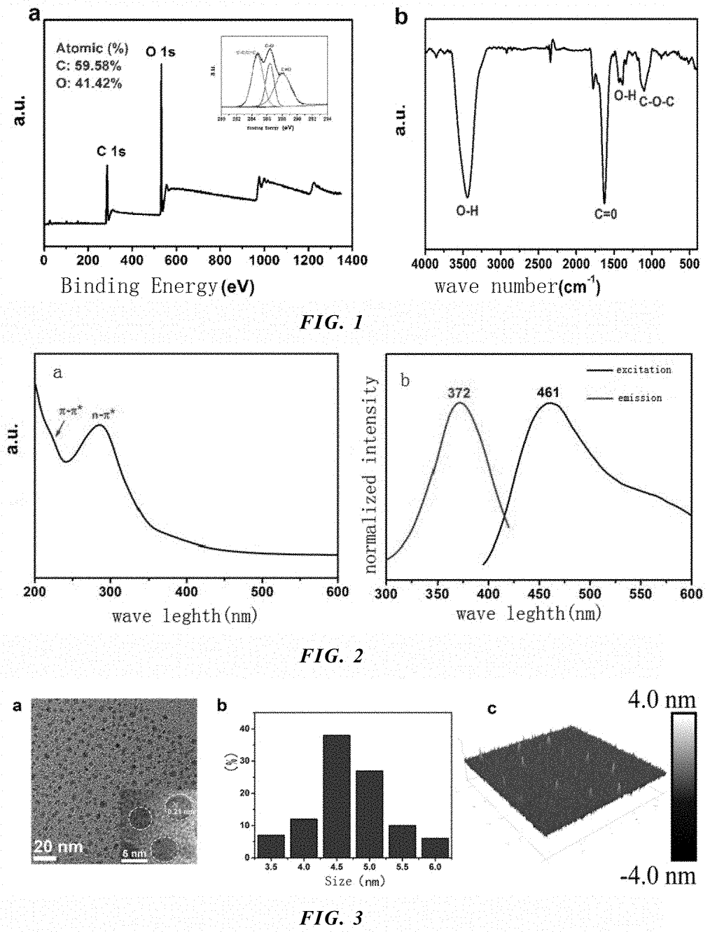

The chemical structure and element composition of CDs were analyzed by FTIR and XPS. From the XPS (FIG. 1a), the CDs mainly contained C and O. From the high-resolution XPS spectrum of C1s, it can be concluded that the three peaks are attributed to C—C, C—O and C═O at 284.8 eV, 286.3 eV and 288.8 eV. FIG. 1b is the FTIR of CDs, it can be seen from the figure that the carbon dots contained hydrophilic functional groups such as —OH and —COOH, which make the CDs have good water solubility.

FIG. 2 detects the optical properties of CDs by ultraviolet-visible absorption spectroscopy and fluorescence spectroscopy. FIG. 2a is the UV-Vis absorption spectrum of CDs. CDs shows two absorption peaks. The absorption peak at 243 nm is due to the transition of CDs π-π*, and the absorption peak at 293 nm is due to the transition of CDs n-π*. From the fluorescence spectrum of the CDs aqueous solution (FIG. 2b), it can be seen that CDs exhibited the strongest emission peak at 461 nm under the excitation of 372 nm light.

TEM and HRTEM images are shown in FIG. 3a, the average hydrated particle size of CDs is about 4.5 nm (shown in FIG. 3b), and the height is about 4 nm shown by AFM in FIG. 3c.

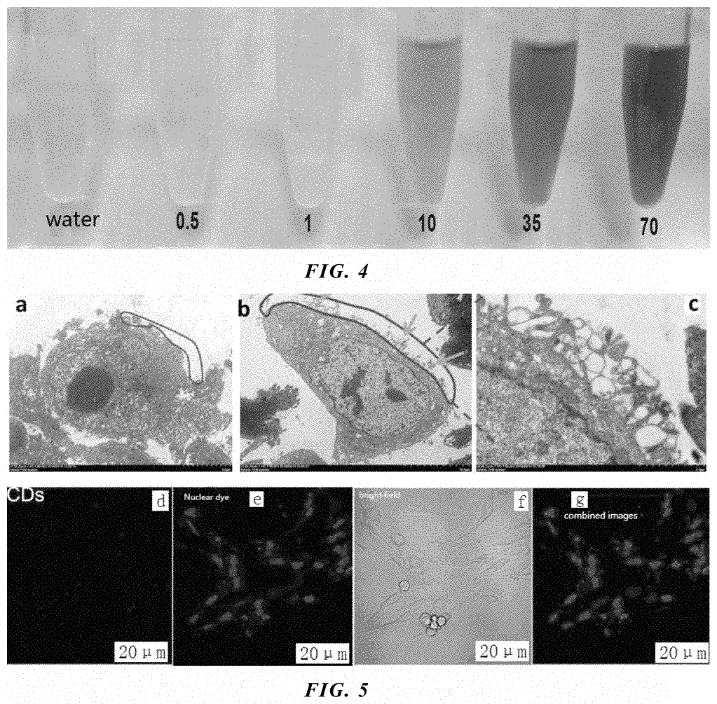

There are photos of the CDs aqueous solution in FIG. 4, wherein the number is the concentration mg/mL. As the concentration increases, the color of CDs aqueous solution changes from light yellow to dark brown, and the highest concentration in the picture reaches 70 mg/mL.

### EXAMPLE 2

CDs Effectively Inhibits the Accumulation of mHtt

The cell sample by transmission electron microscopy: Neuro-2a (N2a) cells and C2N material were incubated in serum-free DMEM medium for 24 hours, then fixed the cells with glutaraldehyde for 10 minutes, scraped the cells and dropped them on a copper mesh, and proceeded 2% Phosphotungstic acid negative staining, in the electron drying oven overnight, and then taken pictures with transmission electron microscope. Protein sample: Dropped the mHtt monomer (100 μM) on the copper mesh and stood for 2 minutes. The filter paper absorbed the excess sample. The biological sample is washed twice with ultrapure water. The sample is negatively stained with 2% Phosphotungstic acid for 2 minutes. Removed the excess Phosphotungstic acid by filter paper absorbed and dried overnight.

CDs and N2a cells were incubated for 12 hours, which confocal laser detection of CDs entering the cell nucleus. Absorbed the culture medium, added Red Dot1 for 10 minutes, and then imaged under a confocal microscope. CDs were excited at 405 nm and Red Dot1 at 640 nm, which combined the images.

CDs are able to enter the cell nucleus, but many materials cannot, or they are excreted in the form of “exosomes” after entering the cells. After N2a cells and C2N materials (the existing nitride graphene nanomaterial) are incubated, many exosomes appearred outside of the cells (Blue arrow). Sees in FIG. 5, the CDs of the present invention are not only “freely diffuse” into the cell cytoplasm but also into the nucleus, and co-localizing with Red Dot 1.

Different with Alzheimer's disease-related toxic protein Aβ, which aggregates outside the cell, Huntington's disease-related mHtt protein aggregates in the cytoplasmic endosome and cell nucleus. Therefore, some nanomaterials can inhibit the aggregation of Aβ peptides to prevent AD, but no therapeutic effect in HD. The results of laser confocal experiments shown that CDs can co-localize with the cytoplasm and nucleus of N2a cells, indicating that CDs can enter the cytoplasm/nucleus. The nuclear function of CDs provides a prerequisite for CDs to inhibit mHtt protein aggregation in the cell (nucleus).

CDs inhibit the accumulation of mHttQ120 peptides: Th T fluorescence experiment was used to detect whether CDs can inhibit the aggregation of mHttQ120. Studies have confirmed that mHttQn (n>35) can aggregate to form fibers rich in β-sheets. ThT is a specific binding β The fluorescence intensity of the dye at a specific excitation/emission wavelength (450/485 nm) reflects the aggregation degree of the peptide. Resuspend the purchased mHtt Q120 (Shanghai Chutide Biotechnology Co., Ltd.) lyophilization powder with 15 μg/100 μL TFA, sonicate for 10 minutes, volatilize TFA in a fume hood to obtain mHtt peptide membrane, and dissolve the peptide with DMSO The membrane was diluted with PBS to 100 μM and aggregated at 37° C. at 300 rpm to obtain mHtt aggregates, which served as the control group. The experimental group was incubated with the same concentration of mHtt and 200 μg/mL CDs solution. The other conditions were the same as the control group. Samples were mixed at different times with 20 μM ThT, and the data was emitted at an excitation wavelength of 450 nm on a microplate reader at a wavelength of 480 nm. Each sample was repeated three times.

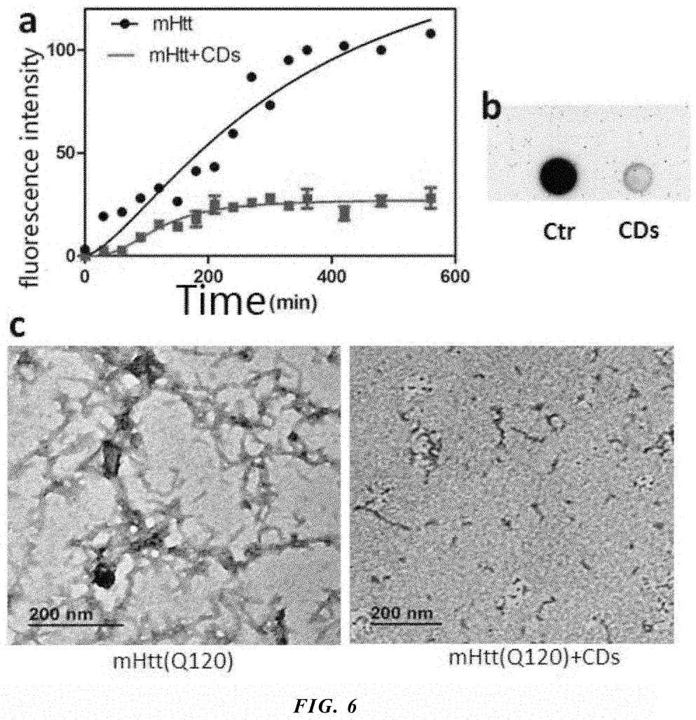

It can be seen from FIG. 6a that mHttQ120 polypeptide can spontaneously aggregate into mature fibers rich in β-sheets in PBS (pH is 7.4, 37° C., 300 rpm), and the fluorescence intensity of Th T increases with time. Compared with the control group, the fluorescence value of mHttQ120 protein aggregates incubated with CDs decreased. At the same time, using the Anti-Amyloid Fibrils antibody that specifically recognizes the fiber conformation, the two groups of samples were subjected to a dot hybridization experiment (1b), which further verified that CDs inhibited the aggregation of mHttQ120 polypeptides to form fibers. In addition, a transmission electron microscope (TEM) was used to observe the morphology of the mHttQ120 aggregate samples incubated with CDs. As shown in FIG. 6(c), the mHttQ120 polypeptide in the control group (without CDs, 0.01M PBS, pH is 7.4) aggregates into a typical fiber structure, while the mHttQ120 polypeptide in the CDs group cannot form a typical fiber structure, which is observed in the field of view It is shorter in length and less dense, showing aggregates in a dispersed state.

Detected the changes of protein secondary structure by CD:

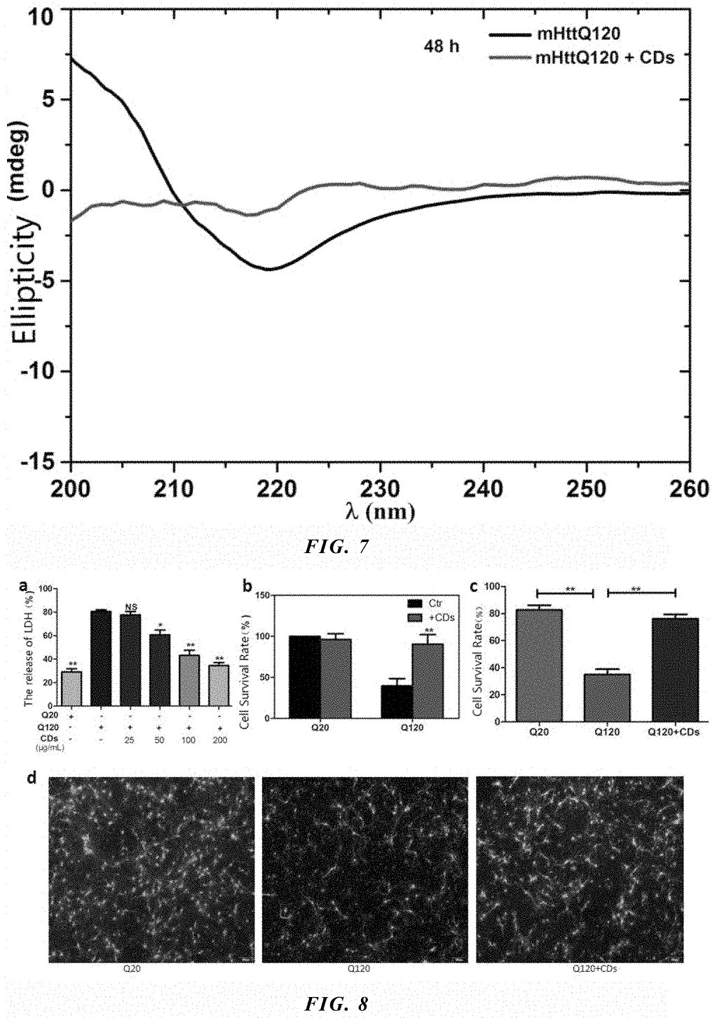

Measure the aggregates, which were the protein secondary structure of mHttQ120+CDs and mHttQ120 with spectropolarimeter J-815. The scanning wavelength was from 200 nm to 260 nm, the spectral width was 2 nm, the scanning speed was 50 nm/min, the response time was 1 s, the measurement temperature was normal temperature, and deducted the equal concentration signal background of material. Each sample was measured 6 times and averaged, and finally the curve was fitted. FIG. 7 shown that the CD spectrum of mHttQ120 polypeptide solution accumulated in PBS for 48 hours shows a typical β-sheet structure (black solid line, there is a negative peak at 220 nm, and a strong positive peak from 200 nm to 210 nm). After the mHttQ120 monomer and CDs (200 μg/mL) were incubated, it was a classic peak shape with irregular curling instead of the typical β sheet peak.

### EXAMPLE 3

CDs Effectively Reduces the Cytotoxicity of mHtt Neuronal Cells

N2a cell model of transiently transfected expressing mHtt: HttExon1Q20/120 (abbreviated Q20/120) plasmid was kept by our laboratory. Cultured N2a cells in DMEM medium of 10% FBS. One day before transfection, planted the cells in the 96-well plate with the density from 3×105/well to 4×105/well. When the cell confluence reached 85%, followed the recommended dosage and steps of Lipofectamine2000™ kit for transfection. Taken two 1.5 ml sterile EP tubes, each added 100 μl Opti-MEM to dilute plasmid DNA and liposomes, the ratio of the two is 1 μg:2 μL, and incubated at room temperature for 5 min after mixing. Mixed the incubated liposomes and plasmids, left them at room temperature and going to incubating for 20 minutes; removed the inoculated cells from the incubator, discarded the complete medium, and added 1 ml Opti-MEM to each well; added the incubated mixture of liposomes and plasmids to each well in proportion, and each well was marked. The 6-well plate was closed to the table and slowly shaken to fully mix; cultured in a incubator with 5% CO2 at 37° C. 4 to 6 hours later, changed with complete medium, cultured for 48 h. It would be used for subsequent experiments. Cells were divided into mHtt20 group (WT, non-toxic polyQ), mHtt120 (toxic polyQ), and CDs groups of various concentrations.

Detection of LDH

Followed the instructions of the LDH kit, treated N2a-mHtt (Q120) with different concentrations of CDs for 48 hours (3 multiple wells per group), added 2% Triton to the positive control group, and added 2% Triton to the negative control group N2a-Htt (Q20); collected the culture solution and centrifuged at 500 g×5 min at 4° C.; taken 100 μL of supernatant to a new 96-well plate (added 100 μL of normal culture solution to the blank control well), and added 100 μL of bottom mixed the substance thoroughly, and incubated for 30 minutes in the dark at room temperature; added the stop solution, and recorded the absorbance value at 490 nm with the microplate reader. LDH release rate (%)=(absorbance of each group-absorbance without cell pores)/(absorbance of positive control group-absorbance without cell pores), the survival rate of the positive control group was set to 100%.

Staining with Trypan Blue

Trypan blue is a kind of cell viability dye that is often used to detect the integrity of cell membranes. When cells are damaged or die, trypan blue can penetrate the denatured cells membrane and bind to the disintegrated DNA to color it. The living cells can prevent the dye into the cells. So it can detect whether the cells are alive. After treating N2a-mHttQ120 with CDs (200 μg/ml) for 48 hours, cells were stained with trypan blue staining solution and then counted directly under a microscope. Cell viability (%)=number of unstained cells/total number of cells observed×100.

Live/Dead Cell (LIVE/DEAD) Staining

The LIVE/DEAD kit is a quick and easy way to distinguish between dead and live cells: Live-Dye dye, a green fluorescent dye that can penetrate cells, is used to stain live cells (Ex/Em=488/518 nm), the red fluorescent dye pyridine iodide (PI) that cannot penetrate the cell membrane stains dead cells (Ex/Em=488/615), and observe the cell death directly under a fluorescence microscope. The LIVE/DEAD experiment was operated in accordance with the kit instructions. After adding the mixed LIVE/DEAD reagent, the cells were incubated in a 37° C. CO2 incubator for 15 minutes, and then transferred to a fluorescence microscope to count live and dead cells. The green represents live cells. Red represents dead cells, cell death rate %=dead cells/(live cells+dead cells).

Because the presence of high levels of mHttQ120 will cause the cells to produce acquired toxicity, the changes of CDs to mHttQ20/120 cytotoxicity were detected. Neuro2a mouse neural cell line (N2a) expressing mHttQ120 plasmid was transiently transfected, and the control group was HttQ20. First, CDs were used to treat N2a-mHttQ20/120 cells for 48 hours to detect the release rate of lactate dehydrogenase in the cell culture medium of each group. The results are shown in FIG. 8. The experimental results showed that CDs inhibited the release of lactate dehydrogenase from N2a-mHttQ120 cells It is concentration-dependent. When the CDs concentration reaches 200 μg/mL, the release rate of lactate dehydrogenase is reduced by 2.5 times. At the same time, N2a-mHttQ20/120 cells treated with CDs (200 μg/mL) were stained with trypan blue for 48 hours. The results showed that CDs can increase the cell survival rate of N2a-mHttQ120 cells, which increased from 40% to 90%. Live/dead cells were stained for each group of cells. A large number of red fluorescent bright spots were seen in mHttQ120 cells. The presence of a large number of dead cells indicated that the overexpression of mHttQ120 protein aggregation is indeed cytotoxic, and the cells of N2a-mHttQ120 cells mixed with CDs The toxicity was significantly reduced, indicating that CDs inhibited the aggregation of mHttQ120, thereby reducing the cytotoxic effect of mHttQ120 aggregation.

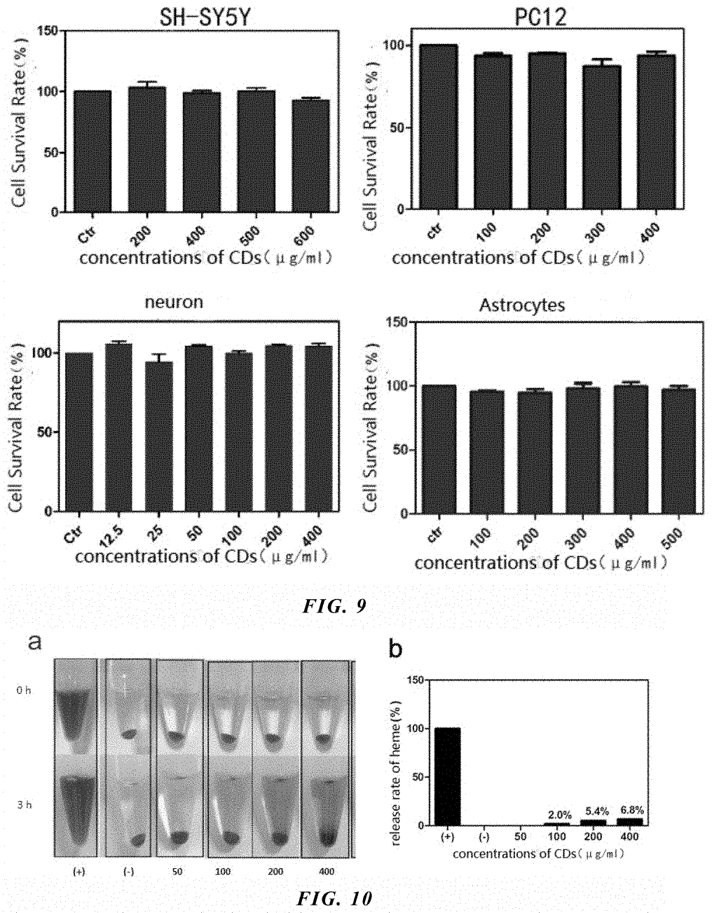

Biocompatibility refers to the compatibility between the material and the host. It is a pervasive theme in nanomedicine research. To evaluate the biocompatibility of nanomedicine/materials should follow the two principles of biosafety and biofunctionality, and the most important indicator of biosafety is non-toxicity. FIG. 9 shows the cell survival rate of different concentrations of CDs, SH-SY5Y, PC12 cells, primary neurons (Neuron), and primary astrocytes after 24 hours of incubation. It can be seen from the figure that when the CDs concentration is 400 μg/mL, the cell survival rate of each group was more than 95%, and there was no significant difference compared with the control group, indicating that CDs within 400 μg/mL were not toxic to cells. The red blood cell hemolysis test was used to detect the destruction of red blood cells by different concentrations of CDs. As shown in FIG. 10, CDs have minimal toxicity to red blood cells, and the hemolysis rate at 400 μg/mL is only 6.8%.

### EXAMPLE 4

Experiment of Animals

Experimental Animals

The model mice of R6/2 (B6CBA-Tg (HDexon1) 62 Gpb/1 J) HD transgenic used in this experiment were purchased from Jackson Labortary Company in the United States. They were raised and bred in an SPF-class animal room, 24 h day and night rotation, room temperature is keep at from 20 to 22° C.; mice have free access to food and water, and the experimental operation follows the experimental animal ethics code; R6/2 transgenic mice are transferred into the first exon of the human HD gene, containing 171 amino acids at the amino terminal, and expressing the amino terminal fragment of Htt It contains 150 glutamine repeats; PCR primers for HD animal genotype identification were purchased from Shanghai Shenggong Biological Engineering Co., Ltd. The primer names are: oIMR1239, oMR1240, β-actinF, β-actinR.

Methods of Animals Grouping and Administration

Mice are divided into four groups: wild-type (WT) intraperitoneal injection of physiological saline (WT+saline) and CDs (1 mg/kg) (WT+CDs), HD transgenic mice intraperitoneal injection of saline (HD+normal saline) and (HD+CDs).

The animals received intraperitoneal injection of CDs or CDs from 5 weeks of age, and the mortality of the mice was evaluated daily.

The analysis of Animals' Behavior

The motor performance was evaluated by the accelerated rotating rod (Stoelting, Ugo Basile, Biological Research apparatus; Varese, Italy) at 5, 8 and 15 weeks of age. At the beginning of each week, mice (n=15) were trained at a slow speed of 4.5 rpm for 30 seconds. Subsequently, three trials were conducted for three consecutive days. In each test, the mouse was placed on a rotating rod at a constant speed of 4.5 rpm for 5 seconds, and then accelerated at a constant rate until a terminal angular velocity of 45 rpm was reached. The incubation period of each mouse falling from the rotating rod was recorded, and the average of three experiments was used for statistical analysis. The wire hanging durability was tested at 9.5 and 16 weeks of age. For this reason, in this experiment, the mouse was placed on a horizontal wire mesh and then gently turned upside down. The time that each mouse stays on the line is recorded. Three experiments were performed on each mouse for three consecutive days, and the average value was used for statistical analysis. The data was analyzed using the hybrid program in software with SAS version 8.2. The results were considered statistically different when P<0.05.

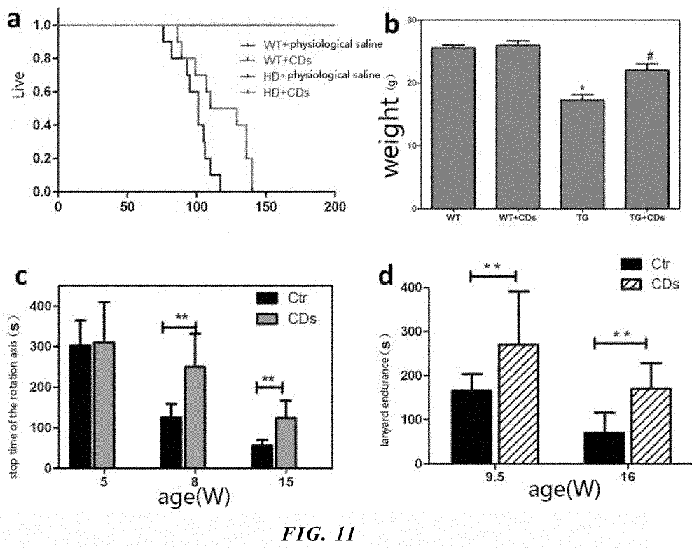

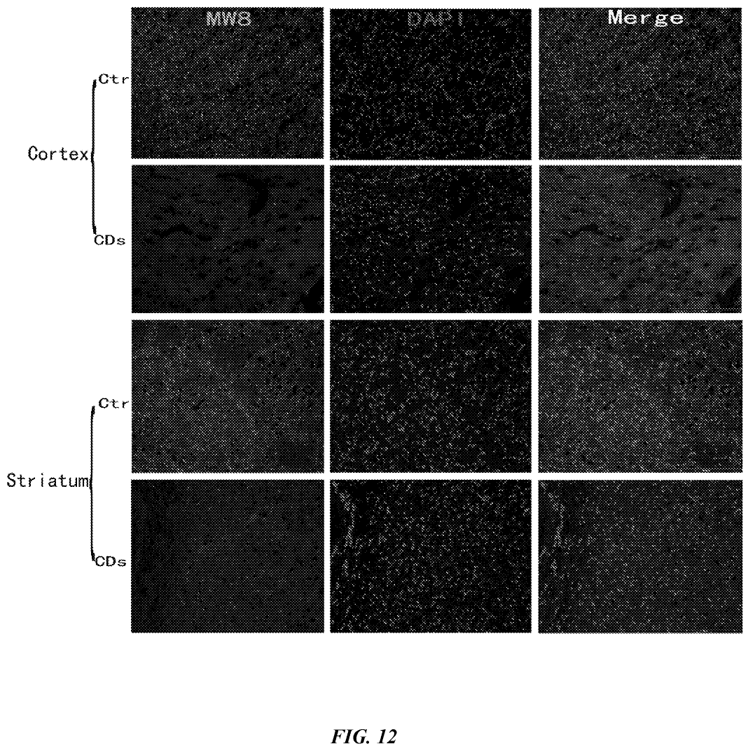

CDs improved the life expectancy, weight loss, and motor function decline of HD transgenic mice: In order to clarify that CDs can inhibit Huntington's disease caused by mHtt aggregation at the animal level, observe the survival rate of CDs in HD transgenic mice (R6/2), Weight and motor function are affected. CDs were injected intraperitoneally from the 5th W of the mouse until the mouse died naturally. The final natural death time of the mouse was recorded and the survival rate was statistically analyzed by the Kaplan-Meier method. Saw FIG. 11. The results are most shown in the normal saline group of HD mice the lifespan is 119.7±7.255 d, and the lifespan of HD mice injected with CDs is 140.54±14.45 d, which meant that CDs treatment can significantly extend the lifespan of HG transgenic mice; among WT mice, CDs treatment does not produce a significant difference in average lifespan (FIG. 11a). Comparison of the body weights of mice in each group of 14 W showed that the weights of the WT mice injected with normal saline and CDs were 24.56±2.3 g and 26.37±2.9 g, respectively; the weight of the HD mice injected with normal saline was 17.32±0.6 g, while the weight of HD mice injected with CDs in the abdominal cavity was 22.19±1.6 g, indicating that CDs can significantly inhibit the weight loss of HD mice (FIG. 11b). The survival rate and body weight of the mice in the WT group and CDs groups were tested There are no significant differences. In order to test the exercise balance ability and grasping power of HD mice, a rotating shaft test (rotating rod test) was performed on HD mice (physiological saline group and CDs administration group). The results show that CDs treatment can extend the residence time of HD mice on the rotating rod apparatus from 56.64±2.3 s to 124.26±6.7 s in the saline group (FIG. 11c). Consistent with the improvement in exercise performance, it was observed that the thread hanging durability of CDs-administered mice was significantly improved at 9.5 and 16 weeks of age. The above results indicate that CDs can significantly improve the behavioral characteristics of HD mice and have a good quality effect on HD mice.

CDs reduce the deposition of mHtt in the brain of HD transgenic mice (R6/2): In order to determine whether CDs reduce the accumulation of mHtt and mHtt in HD transgenic mice, immunohistofluorescence was used to observe the mHtt protein in the brain tissue of each group of HD transgenic mice See FIG. 12 for the aggregation situation. Experimental results shown that CDs treatment can significantly reduce the immunoreactivity of mHtt in the brains of HD mice, which is manifested by the reduction of mHtt (MW8 antibody) positive staining in neuronal nuclei and endosomes; therefore, CDs can be effective at the animal level Inhibit the aggregation of mHtt.

### EXAMPLE 5

Weighed 1.00 g of left-handed vitamin C (L-Vc) and dissolved it into 10 mL of H2O. Ultrasound for 20 minutes to complete dissolution; transfer the dissolved Vc solution to a hydrothermal kettle and reacted at 180° C. for 2 h. After the end, natural cooling to room temperature, then filter the reaction solution with a funnel to remove insoluble particles, and then purified with the 500 to 1000 Da dialysis bag in water, finally obtained the CDs solution was in brown-red; frozen the brown-red CDs solution in the refrigerator at −18° C. for 2 h, and then lyophilization at −80° C. and a vacuum of 10 Pa for 48 h with an Alpha1-4LSCplus RC6 freeze dryer to obtain carbon-based nanomaterial CDs. The obtained carbon-based nanomaterial CDs were dissolved in pure water to obtain the aqueous solution of carbon-based nanomaterial CDs. Referred to the previous test method to test the effect of the above CDs on inhibiting mHtt aggregation. It is found that the freezing process has an impact on the effect of carbon-based nanomaterials, indicating that different degrees of lyophilization have an impact on the formation and performance of carbon-based nanomaterials. Refer to Examples In the third method, N2a-mHttQ20/120 cells treated with CDs (200 μg/mL) in this example were stained with trypan blue for 48 hours. The results showed that CDs can improve the cell survival rate of N2a-mHttQ120 cells, which is 81%, which is slightly lower The CDs in Example 1; FIG. 13 shows the TEM results of the CDs in Example 1 inhibiting the aggregation of mHttQ120, which is slightly inferior to the CDs in Example 1.

Comparative

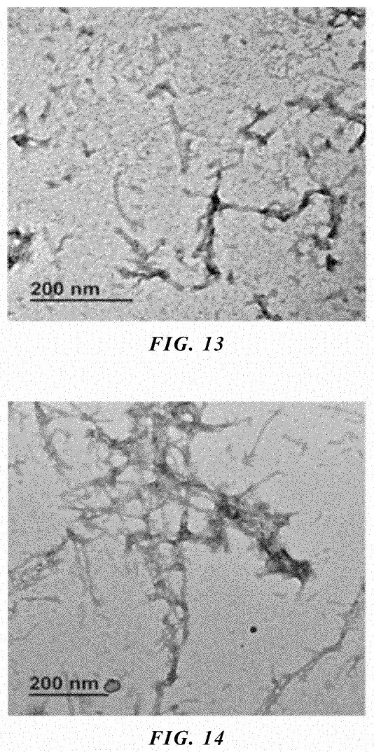

The L-vitamin C in Example 1 was instead of citric acid, in the same way, the water-soluble carbon material can be prepared with the max solubility of 65 mg/mL and the particle size is about 7.5 nm. Referred to the method of Example 3. The comparative carbon material (200 μg/mL) treated N2a-mHttQ20/120 cells for 48 hours and trypan blue staining, and the results showed that it can improve the cell survival rate of N2a-mHttQ120 cells. The cell survival rate is 48%, which is lower than CDs in Example 1. FIG. 14 shows the TEM results of the carbon material inhibiting the aggregation of mHttQ120. It can be seen that the CDs are far inferior to Example 1, and have almost no inhibitory effect on the aggregation of mHttQ120.

In summary, the charge on the surface of nanoparticles, ligand energy, and polypeptide binding ability are all key factors that affect the aggregation of mHtt. For example, the aggregates formed by lysozyme amyloid can be destroyed by nano-gold modified with GSH, but GSH alone does not have this effect; the existing carbon nanomaterials have the disadvantage of poor water solubility, which hinders Their applications in biomedicine and nanomedicine. The present invention has developed carbon-based nanomaterials (CDs) that have the advantages of low cost, extremely small size, good water solubility, high biocompatibility, degradability, and good effects. They are applied to the preparation of anti-HD drugs and found that CDs can inhibit The accumulation of mHtt, cell experiments and animal experiments have found that CDs can alleviate the toxicity of mHtt aggregates to neurons and reduce the damage to synapses, and can improve the exercise ability of HD model mice.

1. An application in the preparation of mHtt aggregation inhibitors or mHtt scavengers; or application of carbon-based nanomaterial in the preparation of drugs for the treatment or alleviation of HD; or application of carbon-based nanomaterial in the removal of mHtt or the inhibition of mHtt aggregation; wherein the carbon-based nanomaterial is prepared from vitamins or quasi-vitamins.

2. According to the application of claim 1, wherein the carbon-based nanomaterial is prepared by heating reaction between vitamins or quasi-vitamins.

3. According to the application of claim 2, wherein the vitamin solution or the quasi-vitamins solution undergoes heating reaction to prepare carbon-based nanomaterial; the concentration of the vitamin solution is 0.1 g/mL; the concentration of the quasi-vitamins solution is 0.1 g/mL.

4. According to the application of claim 2, wherein the heating reaction is at 170° C. to 190° C., for 1.5 h to 2.5 h.

5. According to the application of claim 2, wherein after heating reaction, naturally cooled to room temperature, and filtered; then the filtrate is dialyzed and lyophilization to obtain the carbon-based nanomaterial.

6. According to the application of claim 5, wherein dialysis with 500 to 1000 Da dialysis bag in water; the lyophilization is freezing at −80° C. for 2 h, and then lyophilization at −80° Cwith vacuum of 10 Pa for 48 h.

7. A drug for inhibiting mHtt aggregation or treating HD, wherein comprising the carbon-based nanomaterial; the carbon-based nanomaterial is prepared by heating a vitamin solution or quasi-vitamins solution; the concentration of the vitamin solution is 0.1 g/mL; the concentration of the quasi-vitamins solution is 0.1 g/mL.

8. According to the drug of claim 7, wherein the drug is the aqueous solution of carbon-based nanomaterial.

9. A method for inhibiting the aggregation of mHtt, wherein comprises the follow steps, the aqueous solution of carbon-based nanomaterials and mHtt monomers are incubated to achieve the inhibition of mHtt aggregation.

10. According to the method for inhibiting mHtt aggregation of claim 9, wherein the vitamin solution or the quasi-vitamins solution undergoes heating reaction to prepare carbon-based nanomaterial; the concentration of the vitamin solution is 0.1 g/mL; the concentration of the quasi-vitamins solution is 0.1 g/mL.
